(19) United States
(12) Patent Application Publication
(10) Pub. No.: US 2007/0253865 A1
(43) Pub. Date: Nov. 1, 2007

(54) Sterilization Method and Sterilization Apparatus

(76) Inventors: Ai Tsutsui, Soraku-gun (JP); Kazuro Nishikawa, Higashiosaka-shi (JP); Hisaharu Yagi, Soraku-gun (JP); Yoshihiro Shimizu, Izumi-shi (JP)

Correspondence Address: BIRCH STEWART KOLASCH & BIRCH, PO BOX 747, FALLS CHURCH, VA, 22040-0747, US

(21) Appl. No.: 11/662,202

(22) PCT Filed: Sep. 2, 2005
(86) PCT No.: PCT/JP05/16106

§ 371 Date: Mar. 8, 2007

(30) Foreign Application Priority Data
Sep. 9, 2004 (JP) 2004-262862

(51) Int. Cl. A61L 2/14

(52) U.S. Cl. 422/28; 422/243

(57) ABSTRACT

Disclosed herein are a sterilization method and a sterilization apparatus which are capable of exerting a sterilizing effect on all microorganisms or viruses and which are safe for a living body to be sterilized. The sterilization method includes releasing reactive particles onto microorganisms or viruses to fragment proteins contained in the microorganisms or viruses on condition that nucleic acids contained in the microorganisms or viruses are not disrupted. The sterilization apparatus releases air containing reactive particles that fragment proteins without disrupting nucleic acids to kill microorganisms or viruses present in a target.

### TECHNICAL FIELD

The present invention relates to a sterilization method and a sterilization apparatus.

### BACKGROUND ART

In the field of sterilization, there are conventionally known sterilization methods such as application of sterilizing agents, and many of these methods are still practically used today. On the other hand, Japanese Patent Laying-Open No. 07-108056 (Patent Document 1) proposes a technique for sterilizing hands and fingers with ozone having strong sterilizing power, and Japanese Patent Laying-Open No. 62-119885 (Patent Document 2) proposes a technique for killing bacteria adhered to the surface of an object by heating the object itself with far-infrared radiation. In addition, a method for inactivating bacteria by irradiating the bacteria with ultraviolet rays to directly damage the nucleic acids of the bacteria to thereby inhibit the growth of the bacteria is also practically used (see, for example, Japanese Patent Laying-Open No. 09-225458 (Patent Document 3)).
Patent Document 1: Japanese Patent Laying-Open No. 07-108056
Patent Document 2: Japanese Patent Laying-Open No. 62-119885
Patent Document 3: Japanese Patent Laying-Open No. 09-225458

### DISCLOSURE OF THE INVENTION

Problems to be Solved by the Invention

However, among these conventional sterilization methods, methods using sterilizing agents have a problem in that currently-used sterilizing agents cannot completely and widely exert their sterilizing effect on all bacteria or viruses, and that the concentration at which a sterilizing agent exerts its sterilizing effect depends on the kind of agent used and the time of exposure to a sterilizing agent required to achieve sterilization also depends on the kind of agent used. In addition, such methods using sterilizing agents also have a problem in that, depending on the kind of agent used, skin allergy is caused or physiological discomfort is caused by the smell of a sterilizing agent (see Patent Document 1).

It is known that ozone used in the sterilization method described in Patent Document 1 is a substance having strong sterilizing power. However, the sterilization method using ozone has a problem in that ozone does not stay in a target area to be sterilized but floats around the target area due to its long lifetime, and then accumulates in the air. In addition, -there is a fear that such high-concentration ozone problematically produces toxicity when inhaled by mistake.

Further, in the method of using ultraviolet irradiation described in Patent Document 3, it is also known that when microorganisms inactivated by ultraviolet rays are irradiated with visible light or near-ultraviolet rays, their damaged portions are repaired by a photoreactivating enzyme so that the microorganisms recover their lost function, and this phenomenon is called photoreactivation. Therefore, when the dose of ultraviolet irradiation is determined, it is necessary to allow for photoreactivation to obtain a desired sterilizing effect (see Patent Document 3). In addition, since ultraviolet rays directly act on nucleic acids, it cannot be denied that there is a possibility that human nucleic acids are damaged to cause carcinogenesis action. The sterilization method using infrared rays described in Patent Document 2 is effective when sterilizing an object which can be heated to a high temperature, but cannot be used for a human body because of the risk of skin burns.

The present invention seeks to solve the above problems, and it is therefore an object of the present invention to provide a novel sterilization method and a sterilization apparatus which are capable of exerting a sterilizing effect on all microorganisms or viruses and which are safe for a living body to be sterilized.

Means for Solving the Problems

The present invention is directed to a sterilization method including releasing reactive particles onto microorganisms or viruses to fragment proteins contained in the microorganisms or viruses on condition that nucleic acids contained in the microorganisms or viruses are not disrupted.

The present invention is also directed to a sterilization method including releasing reactive particles onto an affected area or mucosal area of an animal to fragment proteins contained in microorganisms or viruses present in the affected area or mucosal area on condition that nucleic acids contained in the microorganisms or viruses are not disrupted.

In this regard, it is preferred that the proteins are fragmented on condition that nucleic acids contained in cells of the affected area or mucosal area of the animal are not disrupted.

In any of these sterilization methods according to the present invention, it is preferred that fragmentation of the proteins causes any one of reactions selected from oxidation, reduction, hydrolysis, and addition reaction to occur in the proteins.

Further, in these sterilization methods according to the present invention, it is also preferred that the reactive particles naturally disappear in the air.

It is also preferred that the reactive particles are at least one kind selected from plasma, ion, and radical.

The present invention also provides a sterilization apparatus which releases air containing reactive particles that fragment proteins without disrupting nucleic acids to kill microorganisms or viruses present in a target.

In this regard, the target is preferably an affected area or mucosal area of an animal, and the animal is particularly preferably a human.

Further, in the sterilization apparatus of the present invention, it is also preferred that the reactive particles have the property of causing any one of reactions selected from oxidation, reduction, hydrolysis, and addition reaction to occur in the proteins.

Furthermore, it is also preferred that the reactive particles naturally disappear in the air.

Moreover, it is also preferred that the reactive particles are at least one kind selected from plasma, ion, and radical.

Moreover, it is also preferred that the sterilization apparatus according to the present invention includes: release means having an air channel, through which the air containing reactive particles flows, for releasing the reactive particles onto the target; and control means for controlling the velocity of flow of the air containing reactive particles in the air channel where the air is generated.

Moreover, it is also preferred that the sterilization apparatus according to the present invention further includes means for adding liquid fine particles to the air containing reactive particles.

Moreover, it is also preferred that the control means of the sterilization apparatus according to the present invention controls the velocity of flow of the air containing reactive particles on the basis of information detected by a sensor for detecting a region of the target.

Moreover, it is also preferred that the air channel of the sterilization apparatus according to the present invention has an elastic member at the tip thereof Moreover, it is also preferred that the control means of the sterilization apparatus according to the present invention has a timer for controlling the time during which the air containing reactive particles flows through the air channel.

Effects of the Invention

The sterilization method and apparatus according to the present invention make it possible to effectively and safely kill microorganisms or viruses. Further, the sterilization apparatus according to the present invention makes it also possible to effectively and safely sterilize an affected area or mucosal area of an animal without damaging nucleic acids, and therefore can be used for a human body without fear of causing cancer or the like. Such a sterilization apparatus can greatly contribute not only to application to the field of medical treatment but also to prevention of in-hospital infection.

### BRIEF DESCRIPTION OF THE DRAWINGS

FIG. 1 is a schematic diagram showing one preferred example of a sterilization apparatus 1 according to the present invention;

FIG. 2 is a simplified diagram showing an electric discharge means 2 to be preferably used in a sterilization apparatus according to the present invention;

FIG. 3 is a schematic diagram showing a sterilization apparatus 31 used in an evaluation test;

FIG. 4 is a graph showing the result of Experimental Example 1;

FIG. 5 is a graph showing the result of a test performed on Penicillium chrysogenum in Experimental Example 2;

FIG. 6 is a graph showing the result of a test performed on Stachybotrys chartarum in Experimental Example 2;

FIG. 7 is a graph showing the result of a test performed on Asperigillus versicolor in Experimental Example 2;

FIG. 8 is a graph showing the result of a test performed on Penicillium camambertii in Experimental Example 2;

FIG. 9 is a graph showing the result of a test performed on Cladosporium herbarum in Experimental Example 2;

FIG. 10 is a photograph showing the result of a test performed on Asperigillus versicolor and the result of a test performed on Cladosporium herbarum in Experimental Example 3;

FIG. 11 is a photograph showing the result of Experimental Example 4;

FIG. 12 is a photograph showing the result of Experimental Example 5;

FIG. 13 is a graph showing the result of a test performed on Enterococcus malodoratus in Experimental Example 6;

FIG. 14 is a graph showing the result of a test performed on Staphylococcus chromogenes in Experimental Example 6;

FIG. 15 is a graph showing the result of a test performed on Micrococcus roseus in Experimental Example 6; and

FIG. 16 is a graph showing the result of a test performed on Sarcina flava in Experimental Example 6.

### DESCRIPTION OF THE REFERENCE SIGNS

1 sterilization apparatus 2 electric discharge means 3 release means 4 housing 4a opening of housing 6 voltmeter 7 control means 8 liquid addition means 9 tank 10 target 13 timer 22 dielectric body 23 discharging electrode 24 opposed electrode 25 power supply

### BEST MODE FOR CARRYING OUT THE INVENTION

The present invention is a sterilization method including releasing air containing reactive particles onto microorganisms or viruses to fragment proteins contained in the microorganisms or viruses on condition that nucleic acids contained in the microorganisms or viruses are not disrupted. In the present invention, the phrase “to fragment proteins” refers to structural separation or decomposition of proteins caused by cleavage of molecular bonds in the proteins, and includes decomposition involving chemical modification. Fragmentation of proteins causes a change in their initial molecular weights, and therefore the proteins lose their inherent physical properties and function, thereby enabling a reduction in microorganisms or viruses containing the proteins, generation of amino acids and the like. In the sterilization method according to the present invention, the proteins are fragmented on condition that nucleic acids are not disrupted. In this regard, the term “nucleic acids” refers to DNA or RNA, and includes single and double stranded DNA or RNA.

According to the sterilization method of the present invention, cell membrane proteins are broken down, which causes cell membrane dysfunction resulting in loss or reduction of the ability of microorganisms, such as bacteria and fungi, and viruses to grow. In this regard, it is to be noted that by simultaneously applying a condition that nucleic acids are not disrupted, it is possible to prevent mutation of nucleic acids, thereby reducing the possibilities of development of new toxicity of microorganisms, such as bacteria and fungi, or viruses, development of allergy, and evolution of resistance to materials having sterilizing properties, that is, it is possible to reduce the risk of biohazard caused by emergence of organisms having new properties to a low level.

In the case of a conventional sterilization method using a sterilizing agent, a sterilizing effect depends on the concentration of a sterilizing agent used or the time of exposure to a sterilizing agent. However, unlike such a conventional method, the sterilization method according to the present invention can stably exert a sterilizing effect on a wide variety of microorganisms or viruses, and does not cause a problem such as skin allergy. Further, another conventional method using ozone may cause a problem that ozone accumulates in surrounding space and produces toxicity, whereas the sterilization method according to the present invention does not cause such a problem. Furthermore, unlike a method involving ultraviolet irradiation, the sterilization method according to the present invention does not cause a problem of photoreactivation of inactivated microorganisms as will be described later, and does not adversely affect nucleic acids, thereby eliminating the fear of carcinogenesis action on a human body. Moreover, unlike a method involving infrared rays irradiation, the sterilization method according to the present invention does not cause a burn on the skin of a human body.

The sterilization method according to the present invention can also be preferably used to sterilize an affected area or mucosal area of an animal. That is, the present invention also provides a sterilization method including releasing reactive particles onto an affected area or mucosal area of an animal to fragment proteins contained in microorganisms or viruses present in the affected area or mucosal area on condition that nucleic acids contained in the microorganisms or viruses are not disrupted. The animal, to which the sterilization method according to the present invention can be applied, is a human or an animal other than a human. Examples of such an animal other than a human include dogs, cows, cats, pigs, monkeys, rabbits, rats, and birds. Examples of a target to be sterilized include mucous membranes (for example, eyes, mouth), damaged areas in, for example, hands, legs, body, and face, and diseased areas in these parts.

According to the sterilization method of the present invention, the proteins are preferably fragmented on condition that nucleic acids contained in cells of the affected area or mucosal area of the animal are not disrupted. As described above, according to the sterilization method of the present invention, since a condition that nucleic acids contained in the microorganisms or viruses are not disrupted is applied, there is a high possibility that nucleic acids of the animal present in a region, onto which the reactive particles are released, are not adversely affected either, thereby reducing the risk of mutation of the animal cells or cancer development.

The reactive particles used in the sterilization method according to the present invention refer to atoms or molecules physically or chemically transferred to a higher energy state. As a method for producing such reactive particles, excitation of electrons by electric field, collision with charged particles accelerated by an electric field or the like, photoexcitation, application of kinetic energy and the like can be used. Further, the reactive particles have the property of causing a chemical reaction to occur in organic chemical substances present around the reactive particles, and therefore refer to particles which can directly or secondarily perform an action, such as oxidation, reduction, hydrolysis or addition reaction, on proteins to fragment (that is, decompose) the proteins to thereby change the function of the proteins. Specific examples of such reactive particles include plasma, ion, radical, nitrogen oxides (NO, NOₓ), sulfur oxides (SOₓ), hydrocarbons, hydrogen oxides (H₂O₂, HO₂), accelerated electrons, accelerated protons, atomic hydrogen, and high-energy atoms or molecules released from radioactivity. Among them, air containing at least one kind of reactive particles selected from plasma, ion, and radical is preferably used, and air containing, as reactive particles, positive ions and negative ions is particularly preferably used. In this regard, it is to be noted that there is a case where ozone is produced as a by-product when the reactive particles are generated using the above-described method for producing reactive particles. It is preferred that the concentration of ozone contained in the air containing reactive particles is as low as possible because the lifetime of ozone is long and therefore remains for a long period of time. However, the air containing reactive particles may contain a slight amount of ozone unless the ozone adversely affects a human body or the like in the surrounding.

The reactive particles of at least one kind selected from plasma, ion, and radical can be produced by, for example, electric excitation, and have a relatively short lifetime. Therefore, such reactive particles rapidly fragment proteins contained in the microorganisms or viruses and then disappear in a short period of time. Accordingly, these reactive particles can exert a great sterilization effect on a target, onto which the reactive particles are to be released, without exerting a large influence on the outside of the target. Further, there is no necessity to consider an influence on the outside of the target even under conditions where air containing these reactive particles leaks outside the target.

Examples of the positive ions and/or negative ions include H₃O⁺(H₂O)ₙ (where n is 0 or a natural number) and/or O₂⁻(H₂O)ₘ (where m is 0 or a natural number). It is preferred that positive ions are mainly composed of H₃O⁺(H₂O)ₙ and negative ions are mainly composed of O₂⁻(H₂O)ₘ. This is because these ions can be generated from oxygen or moisture contained in the atmosphere, which reduces loads on environment, and these both positive and negative ions react with each other to easily produce active species having higher activity such as hydrogen peroxide (H₂O₂), hydrogen dioxide (O₂H), and hydroxy radicals (.OH).

For example, molecules of oxygen (O₂) and water (H₂O) contained in the air receive energy from plasma produced by creeping discharge on the surface of a discharge electrode, and are therefore changed into reactive particles. It is to be noted that both positive and negative ions are mainly generated by electric discharge of an ion generator. Usually, both positive and negative ions can be simultaneously generated by alternately applying positive and negative voltages, and then can be released into the air. However, a method for generating both positive and negative ions to be employed in the present invention is not limited to such a method. For example, only either positive or negative ions may be first generated by applying only either a positive or a negative voltage. In this case, ions charged with a polarity opposite to that of the ions, which have been already released, are subsequently generated by applying a voltage opposite in polarity to the voltage that has been already applied. In this regard, it is to be noted that the applied voltage required to generate both positive and negative ions depends on the structure of an electrode, but can be in the range of 3.0 to 5.5 kV, preferably in the range of 3.2 to 5.5 kV.

The composition of both positive and negative ions generated by electric discharge from oxygen molecules and/or water molecules present on the surface of an electric discharge device is as follows. As a main positive ion, H₃O⁺(H₂O)ₙ (where n is 0 or a natural number) is formed by clustering of water molecules, present in the air, around a hydrogen ion (H⁺), generated by electrolytic dissociation of water molecules present in the air due to plasma discharge, by solvation energy. On the other hand, as a negative ion, O₂⁻(H₂O)ₘ (where m is 0 or a natural number) is formed by clustering of water molecules, present in the air, around an oxygen ion (O₂⁻), generated by electrolytic dissociation of oxygen molecules or water molecules present in the air due to plasma discharge, by solvation energy.

Both the positive and negative ions released into space surround bacteria and the like, and then undergo a chemical reaction represented by the following formulas (1) and (2) on the surface of the bacteria to produce hydroxy radicals (.OH) having strong oxidizing power. These hydroxy radicals break down the cell membrane of the bacteria and the like, and therefore the bacteria and the like lose their ability to grow, thereby enabling effective sterilization.
H₃O⁺+O₂⁻→.OH+H₂O₂ (1)
H₃O⁺+O₂⁻→HO₂+H₂O (2)

When the concentration of both positive and negative ions in a target region where the ions exert their effect is expressed as the total number of both positive and negative ions, it is in the range of 50 particles/m³ to 5,000,000 particles/m³, preferably in the range of 500 particles/m³ to 500,000 particles/m³, more preferably in the range of 5,000 particles/m³ to 50,000 particles/m³. If the concentration of both positive and negative ions is less than 50 particles/m³, there is a fear that a sterilization effect cannot be sufficiently obtained. On the other hand, if the concentration of both positive and negative ions exceeds 5,000,000 particles/m³, there is a possibility that the concentration of ozone produced as a by-product is increased, and there is also a possibility that the concentration of ozone produced as a by-product exceeds a level generally considered safe depending on design conditions. Therefore, it can be considered that in view of the stability of a sterilization apparatus (which will be described later) and the like, such an excessively high concentration of both positive and negative ions is not suitable for use unless there is particular necessity. In this regard, it is to be noted that the number of ions is defined as the number of small ions, and a critical mobility in the air is set to 1 cm²/V·sec.

The presence or absence of such reactive particles in air can be determined by analyzing the gas composition of air by using gas mass spectrometry, gas concentration measurement, color change test, odor test, light emission test, generated sound test or the like. The gas mass spectrometry can be carried out using a conventionally known mass spectrometer. The gas concentration measurement can be carried out using a gas chromatograph or an ion counter. The color change test or odor test can be performed based on a sensory test such as a visual observation test or an olfactometric test, or can be performed using a color-difference meter, an odor sensor or the like. The light emission test or generated sound test can also be performed based on a sensory test such as a visual observation test or an olfactometric test, or can be performed using an absorptiometer, a spectroscope, a photo sensor, an illuminometer, a microphone or the like.

It is preferred that the reactive particles to be used in the sterilization method according to the present invention naturally disappear, and the lifetime thereof (which is defined as the time required for the number of reactive particles to decrease logarithmically with time by a factor of natural logarithm) is preferably in the range of about 0.1 microsecond to 3,000 seconds, more preferably in the range of 1 microsecond to 300 seconds. If the lifetime of the reactive particles is less than 0.1 microseconds, the number of reactive particles is significantly decreased during feeding air so that a sufficient amount of the particles cannot reach proteins contained in microorganisms or viruses. On the other hand, if the lifetime of the reactive particles exceeds 3,000 seconds, there is a possibility that the particles do not disappear, and therefore an increase in the concentration of the reactive particles cannot be suppressed so that stable performance cannot be maintained. It is to be noted that the lifetime of the reactive particles refers to the time required for the number of generated reactive particles to decrease logarithmically with time by a factor of natural logarithm. Such a lifetime of the reactive particles can be determined by, for example, measuring the concentration of ions contained in a certain quantity of air flowing through an air channel provided between an ion counter and electric discharge means (which will be described later) as an ion generator while changing the length of the air channel to compare the thus measured numbers of ions.

When air containing reactive particles having a lifetime in the range of, for example, 0.1 microseconds to 3,000 seconds is used, these reactive particles rapidly react with proteins, and therefore a predetermined sterilization effect can be obtained with stability. Further, when such air is used, the reactive particles do not accumulate in space, and therefore a stable amount of gas can be released onto a target. The reactive particles of at least one kind selected from plasma, ion, and radical have a relatively short lifetime in space, but the lifetime thereof can be appropriately controlled by a well known condition so as to fall within the above range. Further, the reactive particles of at least one kind selected from plasma, ion, and radical have a short lifetime not only when released into space but also when come into contact with solid matter. Therefore, when come into contact with microorganisms or viruses, such reactive particles break down proteins but do not break down nucleic acids, that is, do not change genes. Therefore, these reactive particles can be used for both microorganisms or viruses present in an affected area or mucosal area of an animal and the animal itself without fear of developing cancer.

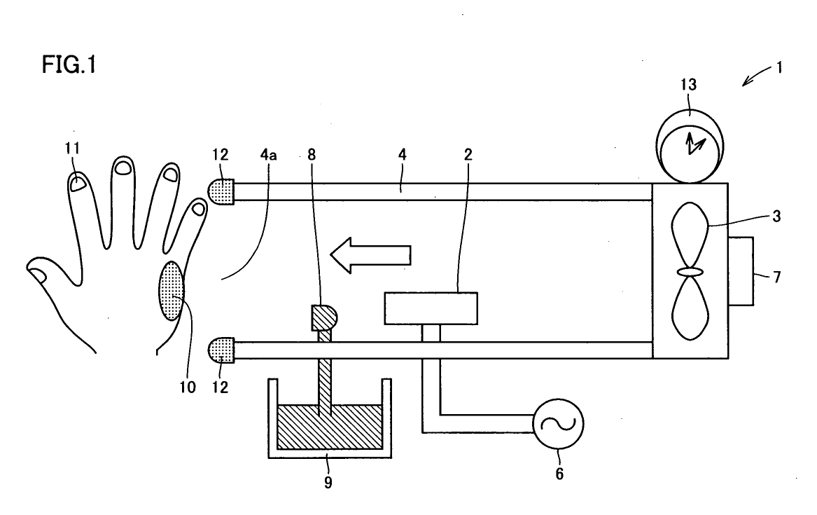

FIG. 1 is a schematic diagram showing one preferred example of a sterilization apparatus 1 according to the present invention. The present invention also provides apparatus (sterilization apparatus) 1 which releases air containing reactive particles that fragment proteins without disrupting nucleic acids to kill microorganisms or viruses present in a target. In the present invention, the target is preferably an affected area or mucosal area of an animal, particularly preferably an affected area or mucosal area of a human. As a matter of course, as described above with reference to the sterilization method according to the present invention, the target may be an affected area or mucosal area of an animal other than a human. Examples of such an animal other than a human include dogs, cats, pigs, monkeys, rabbits, rats, and birds. The sterilization apparatus according to the present invention can effectively and safely sterilize an affected area or mucosal area of an animal without damaging nucleic acids, and therefore can be used for a human body without fear of developing cancer. Such a sterilization apparatus can greatly contribute not only to application to the field of medical treatment but also to the prevention of in-hospital infection.

As described above with reference to the sterilization method according to the present invention, the reactive particles released from the sterilization apparatus according to the present invention preferably have the property of causing any one of reactions selected from oxidation, reduction, hydrolysis, and addition reaction. Further, as described above, the reactive particles preferably have the property of naturally disappearing. More preferably, the lifetime of the reactive particles is in the range of 0.1 microseconds to 3,000 seconds. The reactive particles having such properties disappear without accumulating even when leaked from a human body, and therefore do not unnecessarily break down proteins, thereby eliminating an adverse effect on the human body. Further, the reactive particles are preferably at least one kind selected from plasma, ion, and radical. It is preferred that the sterilization apparatus according to the present invention can kill microorganisms or viruses present in an affected area or mucosal area of a human body with hydroxy radicals generated by the chemical reaction of reactive particles of at least one kind selected from plasma, ion, and radical which have reached the surface of the human body.

The sterilization apparatus according to the present invention basically includes electric discharge means 2 for producing air containing reactive particles, and release means 3 for releasing the reactive particles generated by electric discharge means 2 onto a target. Electric discharge means 2 to be used in the sterilization apparatus according to the present invention is not particularly limited, and a conventional one widely used for generating the air containing reactive particles can be appropriately used. Examples of such electric discharge means 2 include various electric discharge devices such as a creeping discharge device, a corona discharge device, and a plasma discharge device; and one utilizing a device emitting ultraviolet rays or electron rays. The shape and material of an electrode of the electric discharge means are not particularly limited, and a conventionally known one can be appropriately selected.

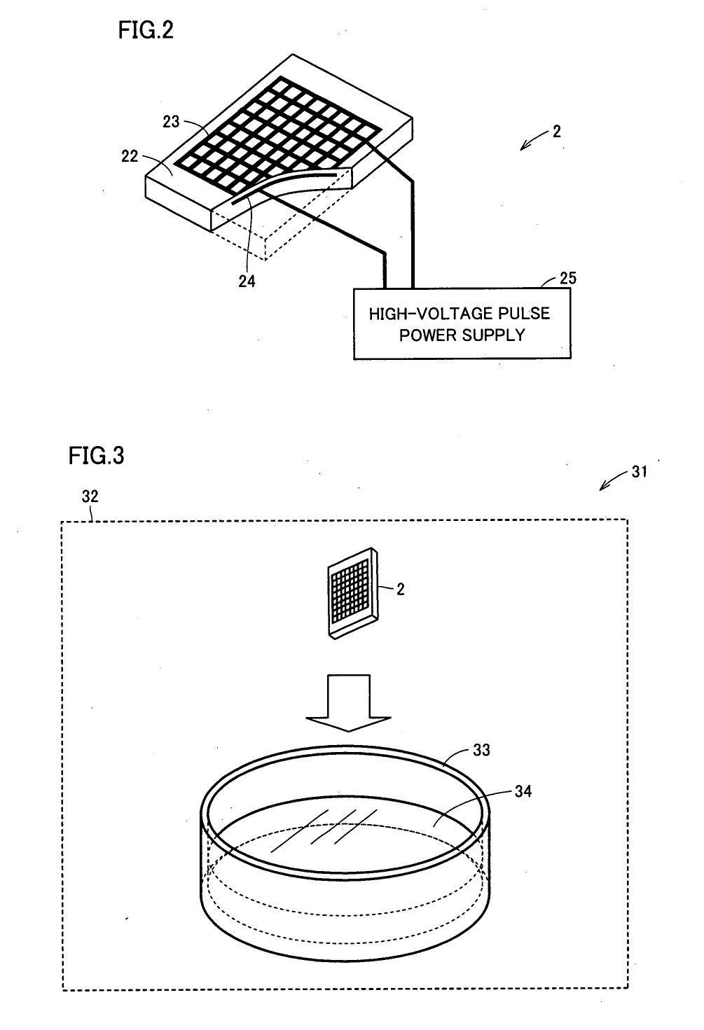

FIG. 2 is a simplified diagram showing electric discharge means 2 to be preferably used in sterilization apparatus 1 according to the present invention. In FIG. 2, a creeping discharger is used as an example of electric discharge means 2. Electric discharge means 2 shown in FIG. 2 by way of example basically includes, for example, a dielectric body 22 having a rectangular cross section, a grid-shaped discharging electrode 23 provided on one surface of dielectric body 22, an opposed electrode 24 implanted in dielectric body 22, and a power supply 25. As dielectric body 22, one made of, for example, alumina and having a size of about 1 cm×3 cm can be preferably used. In electric discharge means 2, discharging electrode 23 and opposed electrode 24 are provided so as to have an appropriate interval (for example, 0.2 mm) therebetween. As power supply 25, a high-voltage pulse power supply can be used. Power supply 25 is electrically connected to discharging electrode 23 and opposed electrode 24. Further, as shown in FIG. 1, a voltmeter 6 is electrically connected to electric discharge means 2.

In a case where a creeping discharge device as shown in FIG. 2 is used, whether ions generated at a certain moment are positively or negatively charged is determined by whether a voltage applied across the electrodes of the discharge device is positive or negative. That is, when a negative voltage is applied across the electrodes, the electrodes are negatively charged and therefore water vapor present in the air is charged to form negative ions, so that a large amount of negative ions are contained in the air. On the other hand, when a positive voltage is applied across the electrodes, water vapor present in the air is charged to form positive ions, so that a large amount of positive ions are contained in the air. More specifically, the high-voltage pulse power supply generates a high pulse voltage with positive and negative polarities (frequency: 60 Hz, peak voltage: about 2 kV), and the high pulse voltage is applied across the electrodes. Alternatively, an alternating voltage may be applied across the electrodes to alternately generate positive ions and negative ions.

Release means 3 of the sterilization apparatus according to the present invention is not particularly limited as long as it can move so as to release air, containing reactive particles generated by electric discharge means 2, onto a target 10. For example, as shown in FIG. 1, release means which is composed of a motor and a fan attached to the shaft of the motor and which has a mechanism that allows the air to be flowed by the fan rotated by driving the motor can be preferably used.

The sterilization apparatus according to the present invention further includes a housing 4 which accommodates electric discharge means 2 and release means 3 described above therein and which has an opening on one side thereof In housing 4, release means 3 is provided so as to be opposed to an opening 4a of housing 4 so that the air can be released out of housing 4 through opening 4a. When sterilization apparatus 1 according to the present invention having such a structure is used, air released by release means 3 is processed by electric discharge means 2 into air containing reactive particles, and the processed air is released in the direction represented by an open arrow in FIG. 1 so that reactive particles contained in the air can collide with target 10 positioned on the opening 4a side of housing 4, thereby rapidly killing microorganisms or viruses present in target 10 or depriving microorganisms or viruses of their ability to grow. In FIG. 1, as target 10, an affected area of a human hand 11 is shown by way of example.

It is preferred that sterilization apparatus 1 according to the present invention has an air channel, through which air containing reactive particles generated by discharge means 2 flows, and control means 7 for controlling the velocity of flow of the air in the air channel. In sterilization apparatus 1 shown in FIG. 1 by way of example, the inner wall of housing 4 also serves as an air channel. In this regard, it is to be noted that the mechanism of controlling the velocity of flow of the air in the air channel is not shown in FIG. 1, but can be achieved by using conventional means appropriately selected. By using sterilization apparatus 1 having such control means 7, it is possible to appropriately control the velocity and quantity of flow of air containing reactive particles according to target 10. Control means 7 can be achieved by using, for example, a central processing unit (CPU) or a microcomputer.

It is to be noted that control means 7 preferably controls the velocity of flow of air containing reactive particles on the basis of information detected by a sensor (not shown in the drawings) for detecting a target region onto which the reactive particles are to be released. This makes it possible to sterilize a target such as an affected area or mucosal area of a human body depending on the conditions of the target. Further, when the release of air is completed, sterilization apparatus 1 is stopped and therefore the air containing reactive particles is not unnecessarily released into space, thereby saving electric power. As the sensor, a conventional sensor can be appropriately used, and examples thereof include sensors using infrared rays or visible light, such as imaging sensors, human body sensors, temperature sensors, and humidity sensors.

It is preferred that the sterilization apparatus according to the present invention further includes means (liquid addition means) 8 for adding liquid fine particles to air containing reactive particles. Sterilization apparatus 1 shown in FIG. 1 by way of example includes liquid addition means 8 provided between electric discharge means 2 and opening 4a of housing 4 for adding liquid fine particles to air containing reactive particles, and a tank 9 provided outside housing 4 for storing a liquid to be fed to liquid addition means 8. Examples of such a liquid include water, tap water, alcohol, sterilizing agents, and mixtures of two or more of them. Among them, water is preferably used. By adding fine particles of water to air containing reactive particles with the use of liquid addition means, it is possible to increase the amount of water molecules present around so that clustering is more likely to occur to form H₃O(H₂O)ₙ (where n is 0 or a natural number) as positive ions and O₂(H₂O)ₘ (where m is 0 or a natural number) as negative ions, and the energy of the solvent is reduced. Therefore, these ions can exist with more stability and therefore exert a higher sterilizing effect. According to such a structure, it is possible to prolong the lifetime of ions to a maximum of about 30 seconds. By applying this condition to the sterilization apparatus according to the present invention, it is possible to prolong the lifetime of ions, thereby allowing release of the ions onto a wide affected area even when the flow of air is weak.

The air channel of the sterilization apparatus according to the present invention preferably has an elastic member at the tip thereof. In sterilization apparatus 1 shown in FIG. 1 by way of example, an elastic member 12 is attached to the periphery of opening 4a of housing 4 whose inner wall also serves as an air channel. Examples of a material for forming elastic member 12 include rubber, sponge, cloth, chemical fiber mesh, and elastic plastics. By providing such elastic member 12 at the tip of the air channel, it is possible to bring elastic member 12 into contact with a target (especially, an affected area or mucosal area of a human body), onto which reactive particles are to be released, without damaging the target when air containing reactive particles is released onto the target. In addition, such elastic member 12 can be detached from the air channel and washed with water, thereby preventing recontamination of the affected area or mucosal area with microorganisms or viruses.

Further, control means 7 of sterilization apparatus 1 according to the present invention preferably includes a timer 13 for controlling the time during which air containing reactive particles flows through the air channel. According to such a structure, it is possible to easily operate sterilization apparatus 1 and to use sterilization apparatus 1 for a wide range of people regardless of their stage of disease.

Hereinbelow, the sterilization method and apparatus according to the present invention will be described with reference to various tests performed by releasing air containing reactive particles through an air channel onto a target, and the data of these tests will be presented. It is to be noted that these tests were performed to evaluate the sterilizing effect of air, containing reactive particles generated by electric discharge in an air channel, on adhesive bacteria.

### EXPERIMENTAL EXAMPLE 1

Test conditions are shown below.

A test method used in Experimental Example 1 is summarized as follows. A bacterium was suspended in a PBS buffer solution (pH 7.4), and the suspension was inoculated onto an agar medium 34 contained in a tray 33. Thereafter, a predetermined treatment (that is, H₃O⁺(H₂O)ₙ (where n is 0 or a natural number) and O₂⁻(H₂O)ₘ (where m is 0 or a natural number) generated by electric discharge means were released as positive ions and negative ions, respectively, and these ions were naturally dispersed on the agar medium) was performed, and then tray 33 was incubated at 37° C. for 72 hours. After the completion of incubation, the number of colony forming units was counted. As a first test, a test for examining the sterilizing effect of electric discharge gas on various adhesive bacteria was performed. As adhesive bacteria, Staphylococcus, Enterococcus malodoratus, Sarcina flava, and Micrococcus roseus were used, and each of these bacteria was inoculated onto an agar medium in such a manner as described above and was then incubated at 37° C. for 8 hours to form colonies.

Then, as shown in FIG. 3, an apparatus 31 including electric discharge means 2, release means (not shown in the drawing), and a housing 32 for accommodating electric discharge means 2 and release means was used to cause electric discharge to generate both positive and negative ions from oxygen molecules and/or water molecules present on the surface of an electric discharge device and release, onto a target, air containing, as reactive particles, hydrogen peroxide (H₂O₂), hydrogen dioxide (HO₂), and hydroxy radicals (.OH) generated by the reactions described above. Housing 32 had a size of 21 cm×14 cm×14 cm. Each tray 33 containing agar medium 34, onto which the bacterium had been inoculated, was placed in housing 32 of sterilization apparatus 31, and the air containing reactive particles was released in the direction represented by an open arrow in such a manner that the ions were spread across the surface of agar medium to expose the bacterium to the air containing reactive particles. The positive ion concentration and negative ion concentration measured on agar medium were both about 3,500 particles/cm³ (which was the concentration of small ions measured at a critical mobility of 1 cm²/V·cm). The concentration of ozone was less than 0.01 ppm. It is to be noted that a fan was not provided in the housing so that the bacterium was exposed to the ions dispersed by natural convection.

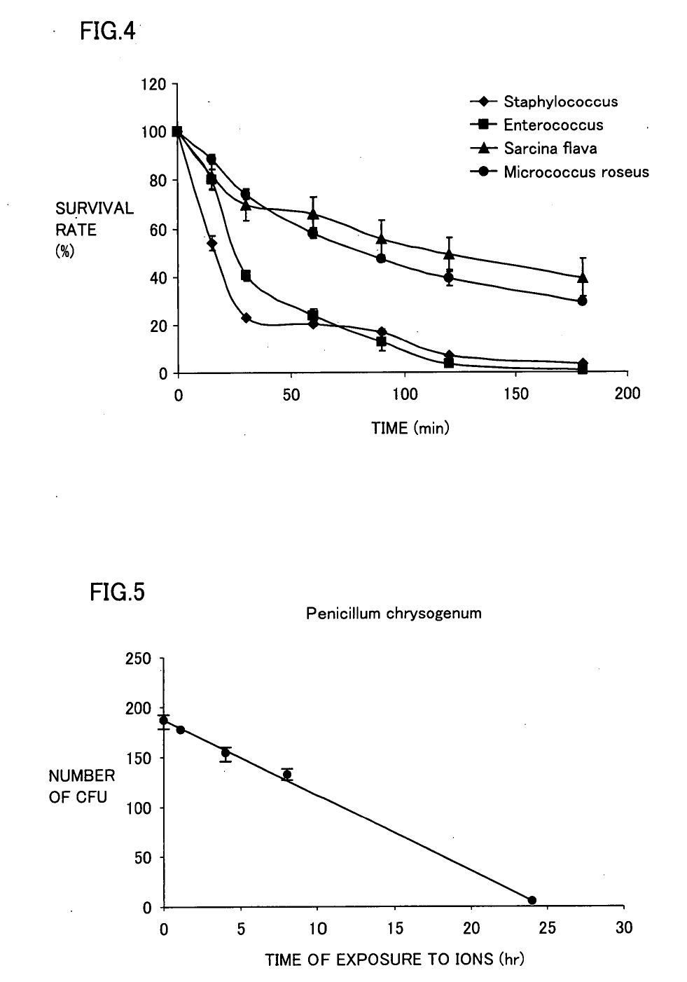

Then, tray 33 was further incubated at 37° C. for 72 hours. After the completion of incubation, the number of colony forming units was counted and the conditions of colonies were observed. The test results are shown in the graph of FIG. 4. As can be seen from FIG. 4, the number of colony forming units (CFU) counted after incubation was smaller when the time for exposing the bacterium to air containing ions as reactive particles was longer. From the result, it has been found that the ions have the effect of sterilizing the adhesive bacteria. Further, as can be seen from FIG. 4, the rate and degree of inactivation were different depending on the species of bacterium. It can be considered that such differences in inactivation rate and inactivation degree are caused by a difference in resistance of cells to plasma, ion, or radical among these bacteria due to variations in their cell structure (that is, cell membrane material, surface and interior conditions of cells, how cells live, and the like).

Thus, the cell walls of these four species of bacteria were compared. The comparison results are shown in Table 1.

TABLE 1

| | Microorganisms | | | |
|---|---|---|---|---|
| | Enterococcus | | Sarcina | Micrococ- |
| | malodoratus | Staphylococcus | flava | cus roseus |
| Capsule | − | − | − | − |
| Pentaglycine | − | + | − | − |
| cross-bridge | | | | |
| structure | | | | |
| Teichoic | Ribitol and | Glycerol | Long-chain | Long- |
| acid | Glycerol | | alcohol | chain |
| | | | | ribitol |
| Metabolic | Fermentation/ | Fermentation/ | Fermentation | Oxidation |
| form | Oxidation | Oxidation | | |
| Oxygen | Facultatively | Facultatively | Aerotolerant | Obligate |
| utilization | anaerobic | anaerobic | | anaerobic |
| | bacterium | bacterium | | bacterium |
| Catalase | − | + | + | + |
| Cytochrome | − | + | − | + |
| Spore | − | − | + | + |
| formation | | | | |
| Pigment | − | +/− | + | + |
| production | | | | |

Table 1 shows the typical constituents of peptidoglycan protein, teichoic acid, and polysaccharides mainly constituting bacteria and the properties of cells. It is to be noted that in Table 1, the symbol “+” indicates that this property is strong, the symbol “−” indicates that this property is weak, and the symbol “±” indicates that this property is neutral.

The items shown in Table 1 will be described below.

A capsule is a membrane made of polysaccharides, and it is said that bacteria having high pathogenicity have a capsule of polysaccharides on the outer side of a peptidoglycan layer.

A pentaglycine cross-bridge structure (5-Gly-cross bridges in cell wall) is one of the structures of a cell wall.

Teichoic acid is a compound of alcohol and phosphate groups, and is contained in a cell wall.

A metabolic form is a method for uptaking substances as raw materials to form constituents of cells and produce energy and releasing by-products.

The item “oxygen utilization” indicates what kind of air environment the bacterium prefers.

Catalase is an enzyme which decomposes hydrogen peroxide into water and oxygen and which functions as an antioxidant.

Cytochrome is one of hemoproteins containing heme iron having oxidation-reduction function.

Spore formation is a property of forming a shell around bacteria.

Pigment production is a property of accumulating/storing self-produced pigment in cells.

The graph shown in FIG. 4 indicates that it took a relatively long time to inactivate Sarcina flava and Micrococcus roseus, whereas Enterococcus malodoratus and Staphylococcus were rapidly inactivated, in spite of the fact that the tests were performed under the same conditions.

In FIG. 4, at the time when 100 minutes have elapsed from the beginning of ion release, the order of inactivation rate from slowest to fastest is Sarcina flava, Micrococcus roseus, Staphylococcus, and Enterococcus malodoratus. Here, the difference in inactivation rate among these bacteria is checked against the properties of these bacteria shown in FIG. 1. As shown in Table 1, Sarcina flava has catalase, exhibits strong properties of spore formation and pigment production, and is further aerotolerant. For this reason, it can be considered that Sarcina flava is highly resistant to highly-reactive substances (that is, ozone, oxygen, ions and the like) present in the air. That is, this case can be considered as a model of the slowest inactivation. Micrococcus roseus has catalase and cytochrome, and exhibits strong properties of spore formation and pigment production. For this reason, it can be considered that Micrococcus roseus exhibits the second slowest inactivation rate, following Sarcina flava.

On the other hand, Staphyrococcus has much catalase and cytochrome, but forms no spore and produces a little pigment. In addition, Staphyrococcus is facultatively anaerobic. For this reason, it can be considered that Staphyrococcus has a lower resistance to highly-reactive substances (that is, ozone, oxygen, ions and the like) than Sarcina flava and Micrococcus roseus.

Enterococcus malodoratus is poor in defense mechanisms such as catalase, cytochrome, spore formation, and pigment production. In addition, Enterococcus malodoratus is facultatively anaerobic. For this reason, it can be considered that Enterococcus malodoratus has a lower resistance to highly-reactive substances (that is, ozone, oxygen, ions and the like) than the other three bacteria. In fact, Enterococcus malodoratus was inactivated most effectively.

The consideration described above suggests that, from the viewpoint of oxygen utilization, aerobic properties make resistance to inactivation higher, and possession of catalase and cytochrome, spore formation, and pigment production make resistance to inactivation higher. In fact, such a tendency was recognized.

The functions of other items shown in FIG. 1, such as capsule, pentaglycine cross-bridge structure, teichoic acid, and metabolic form, cannot be made clear by this test, but it is possible to determine the functions of these items and the extent of their functions by carrying out other control tests.

As described above, by determining the cell structure of each bacterium, it is possible to control the inactivation of cells by the use of air containing reactive particles such as ion, plasma, ozone, radical, or a chemical agent having, for example, oxidation or reduction function. Further, by modeling the relationship between the effect of cell inactivation and cell structure using a predetermined equation, it is possible to calculate the inactivation rate of cells without actually performing an inactivation test as long as the data of the cells, such as cell structure, can be obtained from a database based on the type of cells.

### EXPERIMENTAL EXAMPLE 2

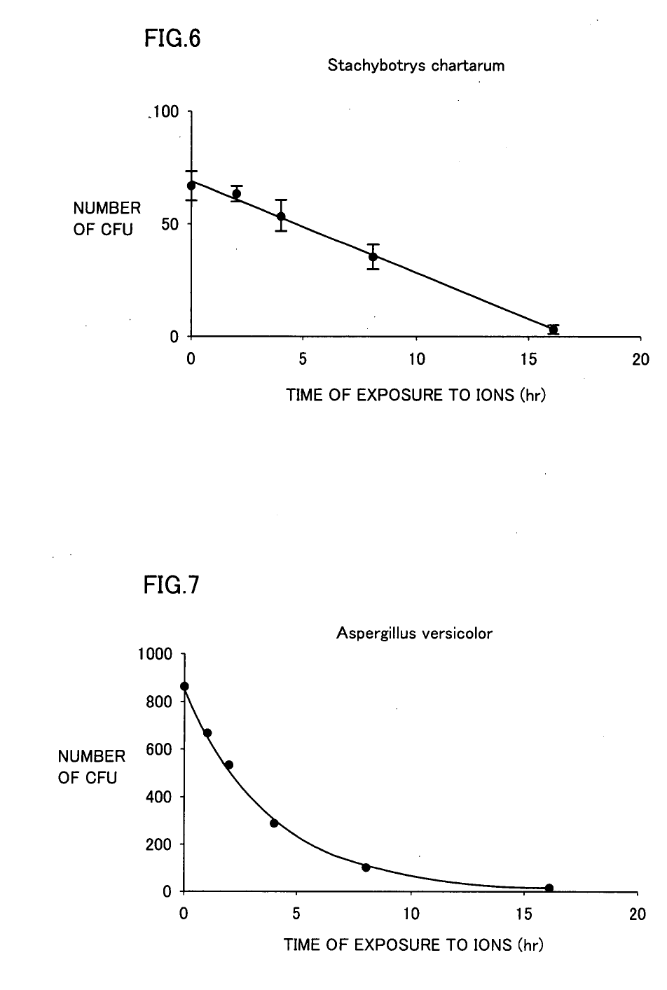

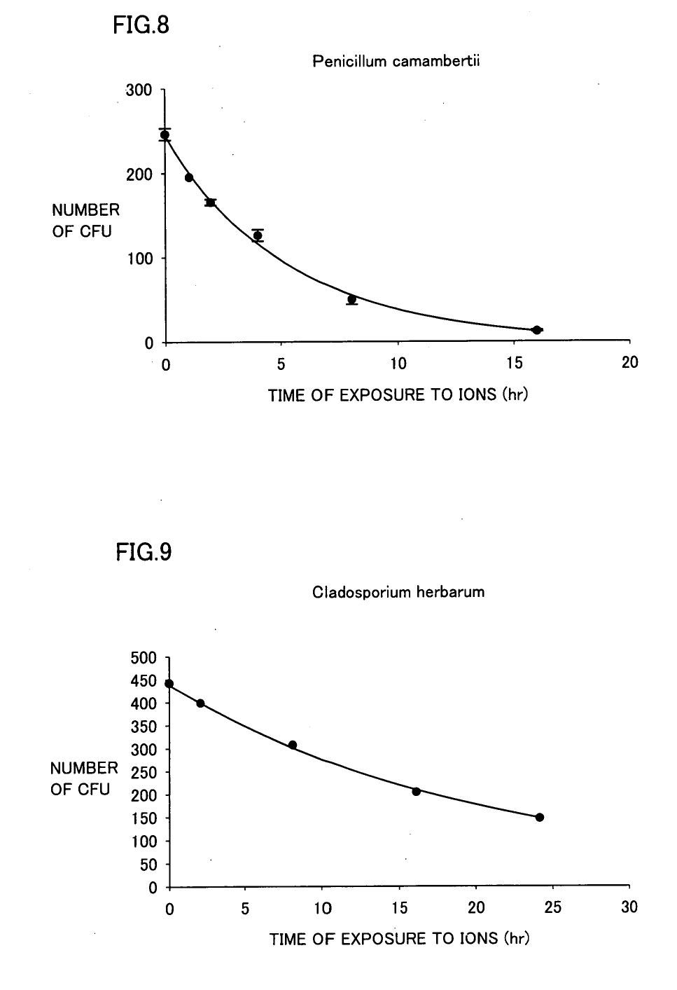

In order to examine the sterilizing effect of air containing reactive particles on fungi, the same test as in Experimental Example 1 was performed on Penicillium chrysogenum, Stachybotrys chartarum, Asperigillus versicolor, Penicillium camambertii, and Cladosporium herbarum. FIGS. 5 to 9 are graphs showing the results of tests performed on Penicillium chrysogenum, Stachybotrys chartarum, Asperigillus versicolor, Penicillium camambertii, and Cladosporium herbarum, respectively. From the results of these tests, it has become clear that also in the case of fungi, the number of colony forming units (CFU) counted after incubation was smaller when the time for exposing each fungus to ions was longer.

### EXPERIMENTAL EXAMPLE 3

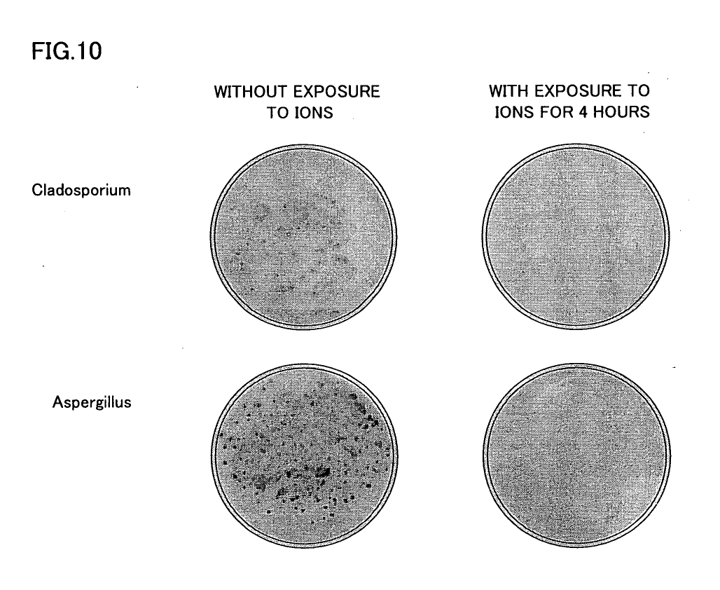

Fungi form spores resistant to thermal shock and physical attack, and therefore there is a fear that when fungi, beginning to form spores, are exposed to ions according to the sterilization method of the present invention, the spores block the ions to prevent decomposition of proteins of the fungi. Thus, Asperigillus versicolor and Cladosporium herbarum, which are very frequently observed in our living environment, were cultured in Petri dishes to once form spores, and then these fungi were exposed to ions for 4 hours in the same manner as in Experimental Example 1 to check to see whether any changes occurred. FIG. 10 is a photograph showing the result of the test performed on Asperigillus versicolor and Cladosporium herbarum in Experimental Example 3. As can be seen from FIG. 10, it was observed that exposure to ions inhibited further spore formation and caused disappearance of fungal colonies, as a result of the above test.

Then, the same test was performed on other fungi. The test results are shown in Table 2. As can be seen from Table 2, also in the case of other fungi, inhibition of spore formation and disappearance of colonies were observed. From the results, it has been found that the ions have the effect of killing gram-positive cocci and fungi, and the adhesive germs of these.

TABLE 2

| Fungi | Effectiveness |
|---|---|
| Aspergillus versicolor | +++ |
| Penicillum chrysogenum | +++ |
| Cladosporium herbarum | +++ |
| Stachybotrys chatarum | ++ |
| Penicillum camambertii | ++ |
| Mucor sp. | +++ |
| Alternaria altemata | +++ |

+++: strong effect,
++: neutral effect

### EXPERIMENTAL EXAMPLE 4

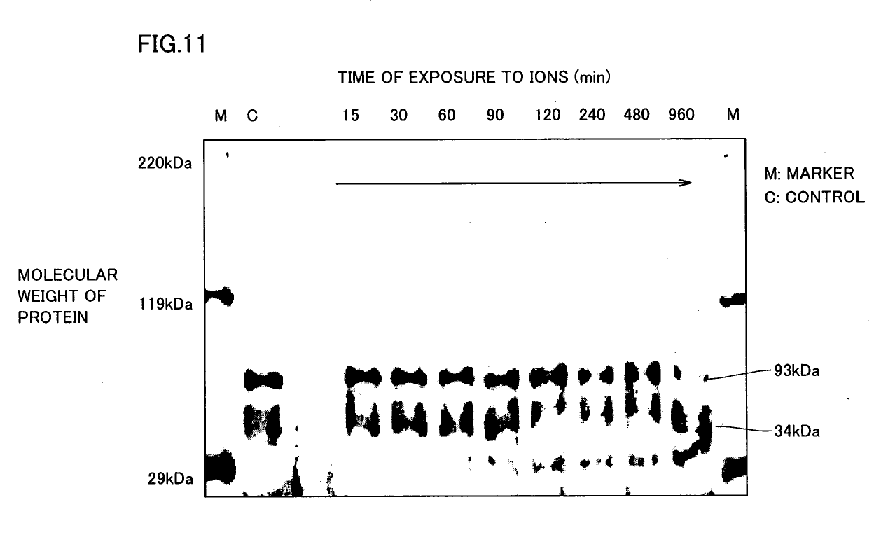

An adhesive bacterium was exposed to air containing ions as reactive particles to observe changes in its proteins. This test was performed in the following manner. Enterococcus malodoratus was inoculated onto a plurality of agar media, and was then exposed to ions in the same manner as in Experimental Example 1 for 15, 30, 60, 90, 120, 240, 480, and 960 minutes, respectively. After the completion of exposure to ions, 15 membrane proteins were extracted from Enterococcus malodoratus, and were then subjected to two-dimensional electrophoresis by SDS-PAGE. FIG. 11 is a photograph showing the result of Experimental Example 4. As can be seen from FIG. 11, a plurality of protein fragments seen as an indication of a pathological phenomenon were observed as a result of exposure to ions. Further, the result shown in FIG. 11 indicates that such fragmentation and aggregation of membrane proteins correspond to the time of exposure to ions, that is, when the time of exposure to ions is longer, the degree of damage of membrane proteins is larger.

The result of Experimental Example 4 can be described by the following probable mechanism. That is, the bacterium inoculated onto the agar medium is singly exposed at the surface of the agar medium at first, but when the bacterium comes into contact with the ions contained in the air, the cell membrane of the bacterium is disrupted so that proteins contained in cells are released to the outside of the cells and cell membrane dysfunction occurs, thus resulting in inactivation of the bacterium (sterilization). It can be considered that FIG. 4 in Experimental Example 1 indicates the result of such action.

### EXPERIMENTAL EXAMPLE 5

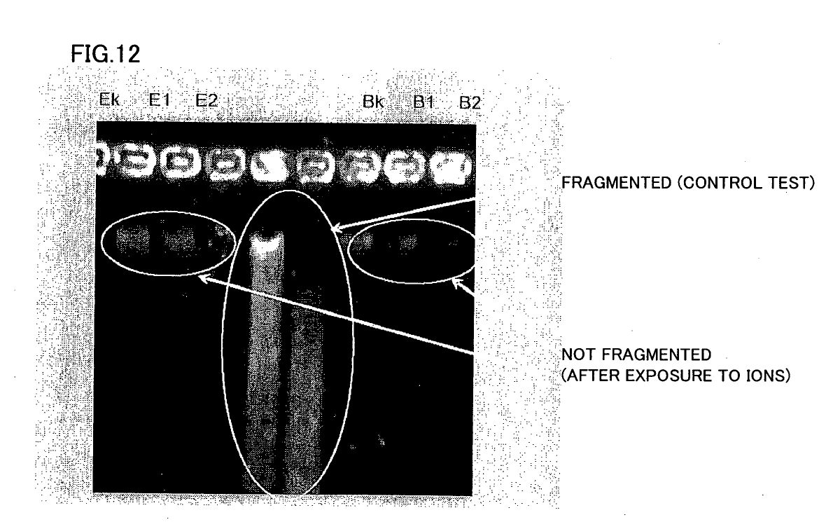

Next, a verification test was performed in the following manner to demonstrate that the ions used in the above tests do not damage DNA and do not cause cancer. Enterococcus malodoratus and Bacillus were exposed to air containing ions in the same manner as in Experimental Example 1 for 0, 1, and 2 hours. After the completion of exposure to ions, DNA was extracted from the bacteria in the usual manner, and was then subjected to electrophoresis. FIG. 12 is a photograph showing the result of Experimental Example 5. In FIG. 12, the lanes correspond to the following:

EK: Enterococcus not exposed to ions

E1: Enterococcus exposed to ions for 1 hour

E2: Enterococcus exposed to ions for 2 hours

BK: Bacillus not exposed to ions

B1: Bacillus exposed to ions for 1 hour

B2: Bacillus exposed to ions for 2 hours

It is to be noted that two middle lanes in FIG. 12 contain, as DNA-fragmentation positive controls, the results of fragmentation by subjecting DNA extracts from Enterococcus and Bacillus to a standard reaction for positive reaction are shown.

As can be seen from FIG. 12, in each lane, DNA exposed to ions revealed a single band, that is, a single stranded DNA was not detected. From the result, it can be said that exposure to ions does not damage DNA and does not involve the risk of causing cancer. It is to be noted that in this test, electric discharge was performed under conditions where H₃O(H₂O)ₙ (where n is 0 or a natural number) and O₂(H₂O)ₘ (where m is 0 or a natural number) were mainly released as positive ions and negative ions, respectively, but reactive particles generated by electric discharge are not limited to these substances. For example, it can be expected that air containing the above-mentioned two or more kinds of substances such as ions (for example, N₂⁺, O₂⁺, NO₂⁻, CO₂⁻) and radicals, will have the same sterilizing effect.

The result of the Experimental Example 5, that is, the findings that exposure of a bacterium to air containing ions disrupts the cell membrane of the bacterium but leaves DNA contained in cells intact can be described as follows. The substances contributing to protein decomposition are positive and negative ions released into space. According to our experiments, the lifetime of these ions varies depending on conditions, but is in the range of about 5 to 30 seconds. This is because these ions are reactive particles, and therefore when colliding with dusts or ions in the air, these ions react with them to disappear. For this reason, it can be considered that when coming into contact with cells, these ions rapidly react with the cells, but do not affect DNA contained in the cells because of their short lifetime. Further, it can be expected that such an effect will be obtained by using particles having a lifetime substantially the same as or shorter than that of ions, such as OH radicals having a lifetime in the air of about 1 microsecond. The above consideration suggests that the use of reactive particles having a relatively short lifetime makes it possible to disrupt a cell membrane while leaving DNA intact.

### EXPERIMENTAL EXAMPLE 6

It is known that bacteria have self-repairing ability and therefore there is a case where, when treatment for killing bacteria is incomplete (for example, the length of time of exposure to ultraviolet rays is too short or the dose of a sterilizing agent is too small), the bacteria come alive and grow. Thus, a test was performed on Enterococcus malodoratus, Staphylococcus chromogenes, Micrococcus roseus, and Sarcina flava to check whether the inactivation of these bacteria by positive and negative ions was irreversible.

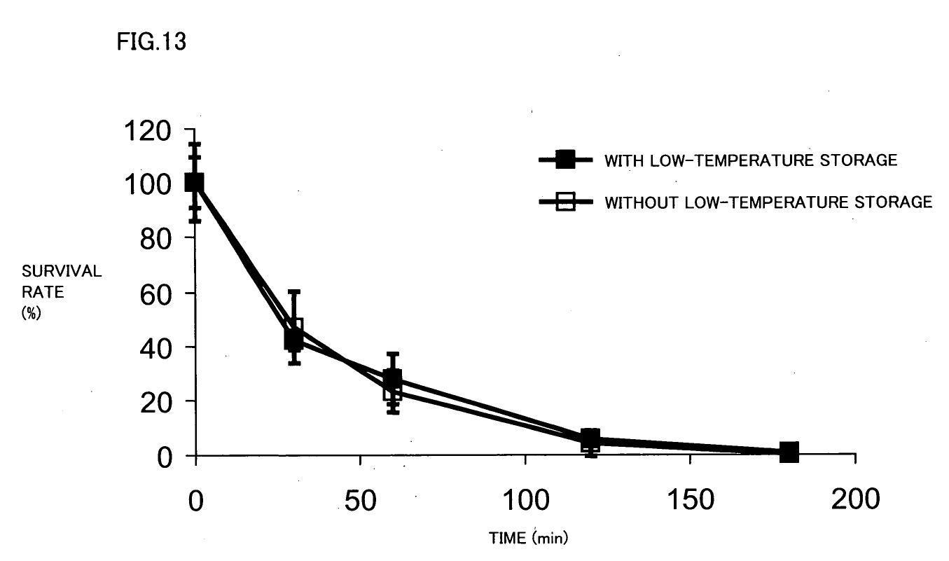

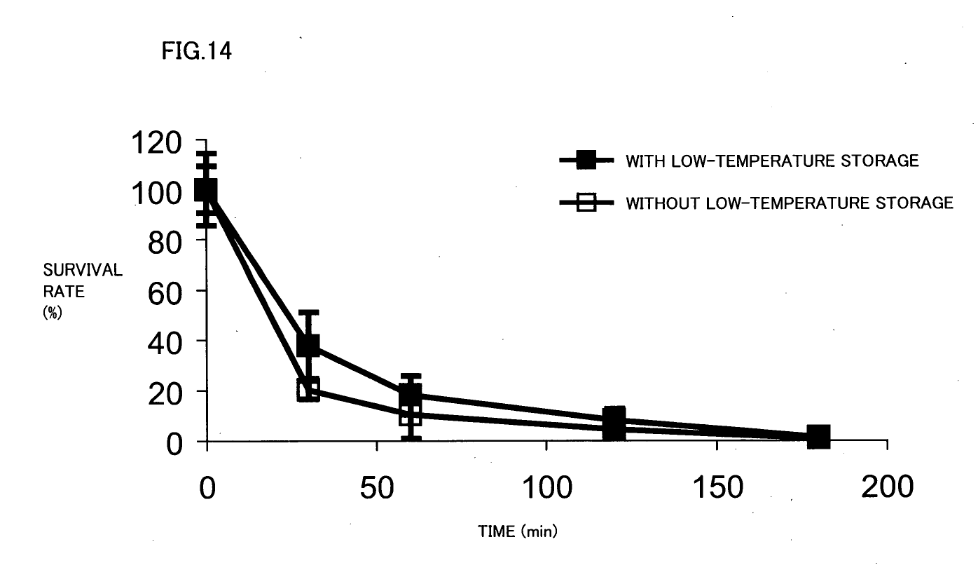

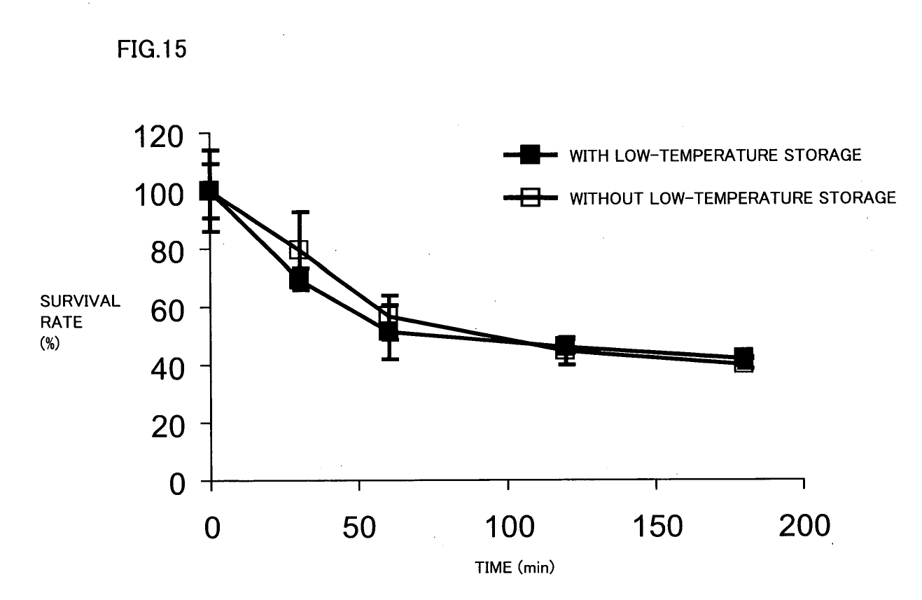

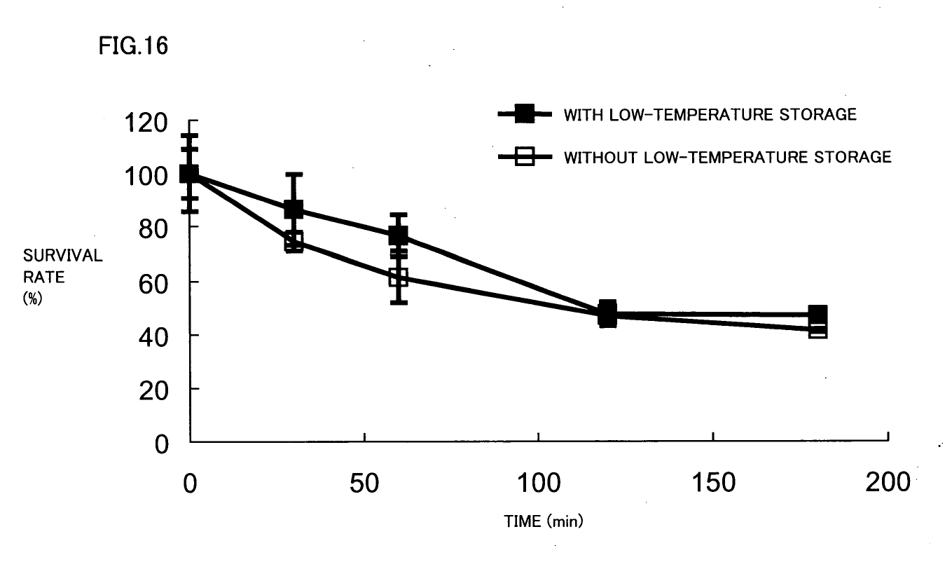

These bacteria were inoculated onto agar media, and were then exposed to air containing both positive and negative ions for 90 minutes in the same manner as in Experimental Example 1. After the completion of treatment with ions, these bacteria were stored at a low temperature of 4° C. for 3 days. The low-temperature storage was carried out to give a recovery time to the bacteria. A change in the number of residual cells exposed to ions was monitored with time in both cases where low-temperature storage was carried out or not carried out. FIGS. 13 to 16 are graphs showing the results of the tests performed on Enterococcus malodoratus, Staphylococcus chromogenes, Micrococcus roseus, and Sarcina flava, respectively. As can be seen from FIGS. 13 to 16, in the cases of all the four kinds of bacteria, there was no significant difference in change in the number of residual cells with time between the cases where low-temperature storage was carried out or not carried out. This means that the bacteria did not recover during low-temperature storage.

Also, another test was performed in the following manner The bacteria were inoculated onto agar media, and were then exposed to air containing both positive and negative ions for 90 minutes in the same manner as in Experimental Example 1. Then, the bacteria were incubated in an incubator at 37° C. for 48 hours to form colonies of the bacteria, and were then further incubated at 37° C. for 21 days to check whether new colonies were formed. As a result, new colonies were not observed even after incubation for 21 days. This means that the bacteria did not recover even under growth conditions.

Still another test was performed in the following manner to examine the influence of a culture medium degraded by exposure to ions on recovery of the bacteria. The bacteria were inoculated onto agar media, and were then exposed to air containing both positive and negative ions. Then, the bacteria were transferred onto culture media not exposed to ions to check whether the bacteria recovered or not. As a result, the bacteria did not recover.

From the results, it has been found that such a method for inactivating bacteria by exposing the bacteria to air containing ions can deprive the bacteria of their self-repairing ability to completely kill them.

Although the present invention has been described and illustrated in detail, it is clearly understood that the same is by way of illustration and example only and is not to be taken by way of limitation, the spirit and scope of the present invention being limited only by the terms of the appended claims.

1-20. (canceled)

21. A sterilization method comprising releasing reactive particles onto microorganisms or viruses to fragment proteins contained in the microorganisms or viruses on condition that nucleic acids contained in the microorganisms or viruses are not disrupted.

22. The sterilization method according to claim 21, wherein fragmentation of said proteins causes any one of reactions selected from oxidation, reduction, hydrolysis, and addition reaction to occur in the proteins.

23. The sterilization method according to claim 21, wherein said reactive particles are at least one kind selected from plasma, ion, and radical.

24. A sterilization apparatus which releases air containing reactive particles that fragment proteins without disrupting nucleic acids to kill microorganisms or viruses present in a target.

25. The sterilization apparatus according to claim 24 which comprises: release means having an air channel, through which the air containing reactive particles flows, for releasing the reactive particles onto the target; and control means for controlling the velocity of flow of the air containing reactive particles in the air channel where the air is generated.

26. The sterilization apparatus according to claim 25, further comprising means for adding liquid fine particles to said containing reactive particles.

27. The sterilization apparatus according to claim 25, wherein said control means contols the velocity of flow of the air containing reactive particles on the basis of information detected by a sensor for detecting a region of the target.

28. The sterilization apparatus according to claim 25, wherein said control means has a timer for controlling the time during which the air containing reactive particles flows through the air channel.
